SEQUENCE LISTING (4 sequences)

SEQ ID NO: 1 - protein, 390 aa, Homo sapiens
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 2 - protein, 429 aa, Homo sapiens
MCPGALWVALPLLSLLAGSLQGKPLQSWGRGSAGGNAHSPLGVPGGGLPEHTFNLKMFLENVKVDFLRSLNLSGVPSQDKTRVEPPQYMIDLYNRYTSDKSTTPASNIVRSFSMEDAISITATEDFPFQKHILLFNISIPRHEQITRAELRLYVSCQNHVDPSHDLKGSVVIYDVLDGTDAWDSATETKTFLVSQDIQDEGWETLEVSSAVKRWVRSDSTKSKNKLEVTVESHRKGCDTLDISVPPGSRNLPFFVVFSNDHSSGTKETRLELREMISHEQESVLKKLSKDGSTEAGESSHEEDTDGHVAAGSTLARRKRSAGAGSHCQKTSLRVNFEDIGWDSWIIAPKEYEAYECKGGCFFPLADDVTPTKHAIVQTLVHLKFPTKVGKACCVPTKLSPISVLYKDDMGVPTLKYHYEGMSVAECGCR

SEQ ID NO: 3 - protein, 498 aa, Homo sapiens
MTVFLSFAFLAAILTHIGCSNQRRSPENSGRRYNRIQHGQCAYTFILPEHDGNCRESTTDQYNTNALQRDAPHVEPDFSSQKLQHLEHVMENYTQWLQKLENYIVENMKSEMAQIQQNAVQNHTATMLEIGTSLLSQTAEQTRKLTDVETQVLNQTSRLEIQLLENSLSTYKLEKQLLQQTNEILKIHEKNSLLEHKILEMEGKHKEELDTLKEEKENLQGLVTRQTYIIQELEKQLNRATTNNSVLQKQQLELMDTVHNLVNLCTKEGVLLKGGKREEEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 4 - protein, 496 aa, Homo sapiens
MWQIVFFTLSCDLVLAAAYNNFRKSMDSIGKKQYQVQHGSCSYTFLLPEMDNCRSSSSPYVSNAVQRDAPLEYDDSVQRLQVLENIMENNTQWLMKLENYIQDNMKKEMVEIQQNAVQNQTAVMIEIGTNLLNQTAEQTRKLTDVEAQVLNQTTRLELQLLEHSLSTNKLEKQILDQTSEINKLQDKNSFLEKKVLAMEDKHIIQLQSIKEEKDQLQVLVSKQNSIIEELEKKIVTATVNNSVLQKQQHDLMETVNNLLTMMSTSNSAKDPTVAKEEQISFRDCAEVFKSGHTTNGIYTLTFPNSTEEIKAYCDMEAGGGGWTIIQRREDGSVDFQRTWKEYKVGFGNPSGEYWLGNEFVSQLTNQQRYVLKIHLKDWEGNEAYSLYEHFYLSSEELNYRIHLKGLTGTAGKISSISQPGNDFSTKDGDNDKCICKCSQMLTGGWWFDACGPSNLNGMYYPQRQNTNKFNGIKWYYWKGSGYSLKATTMMIRPADF

(19) United States
(12) Patent Application Publication
(10) Pub. No.: US 2019/0293661 A1
(43) Pub. Date: Sep. 26, 2019

(54) METHOD OF PREDICTING RESPONSIVENESS OF WET AMD PATIENT TO ANTI-VEGF THERAPY

(71) Applicant: Santen Pharmaceutical Co., Ltd., Osaka-shi, Osaka (JP)

(72) Inventors: Keiichi Shibagaki, Ikoma-shi, Nara (JP); Masanao Toshimori, Osaka-shi, Osaka (JP); Koji Oka, Emeryville, CA (US); Shingo Maeda, Osaka-shi, Osaka (JP); Hiroshi Inoue, Osaka-shi, Osaka (JP)

(73) Assignee: Santen Pharmaceutical Co., Ltd., Osaka-shi, Osaka (JP)

(21) Appl. No.: 16/465,703

(22) PCT Filed: Nov. 30, 2017
(86) PCT No.: PCT/JP2017/043133

§ 371 Date: May 31, 2019

Related U.S. Application Data
(60) Provisional application No. 62/428,823, filed on Dec. 1, 2016.

(51) Int. Cl. G01N 33/68

(52) U.S. Cl. CPC G01N 33/6872; G01N 2800/52; G01N 2800/16; G01N 33/6893

(57) ABSTRACT

The disclosure provides a method of predicting the responsiveness of a wet AMD patient to anti-VEGF therapy comprising (1) determining the level of at least one marker protein selected from the group consisting of TGF-beta, BMP9, angiopoietin-1, and angiopoietin-2 in a blood, plasma or serum sample obtained from the patient, and (2) predicting the responsiveness of the patient to the anti-VEGF therapy with reference to the level determined in step (1), as well as a diagnostic agent for use in the method.

This application claims the benefit of priority of the prior US Provisional application (U.S. Provisional Application No. 62/428,823), the entire contents of which are incorporated herein by reference.

### TECHNICAL FIELD

The disclosure relates to a method of predicting the responsiveness of a wet AMD patient to anti-VEGF therapy or a diagnostic agent for use in the method.

### BACKGROUND ART

One main cause of social blindness in the elderlies is age-related macular degeneration (AMD). Two types of AMD are known: atrophic (dry) AMD, which is associated with geographic atrophy (i.e., atrophy of retinal pigment epithelium and choroidal microvasculature), and exudative (wet) AMD, which is characterized by choroidal neovascularization (CNV) under retina or retinal pigment epithelium. Involvement of vascular endothelial growth factors (VEGFs) in the CNV formation, the main pathology of wet AMD, has been revealed and several anti-VEGF agents have been approved for treating wet AMD.

The anti-VEGF agents are known to exhibit good clinical effects. The agents, however, are expensive and cause large mental and physical burdens on patients because they are administered by intravitreal injection. Furthermore, some patients are refractory to the anti-VEGF therapy. For example, Non-Patent Literature 1 discloses that improvement in visual acuity was displayed by 40% of patients, deterioration by 25% of patients and no change by 35% of patients after treatment with an anti-VEGF agent. No method is available for predicting whether a patient is refractory to the anti-VEGF therapy.

Some anti-VEGF agents are also used for treating a cancer. When the cancer is refractory to the anti-VEGF therapy, use of inhibitors for other molecules modulating angiogenesis might be suggested (Non-Patent Literature 2 and 3). However, the role of the molecules modulating angiogenesis in the development of the refractoriness to the anti-VEGF therapy has not been revealed.

### REFERENCES

### Non-Patent Literature

[Non-Patent Literature 1] Graefes Arch Clin Exp Ophthalmol. 2014; 252(4):647-55.
[Non-Patent Literature 2] Curr Oncol Rep. 2014; 16(2):365.
[Non-Patent Literature 3] J Clin Oncol 2010; 28(15):suppl; abstract 4630.

### SUMMARY OF THE INVENTION

One of the objects of the disclosure is to provide a method of predicting the responsiveness of a wet AMD patient to the anti-VEGF therapy, or a diagnostic agent for use in the method.

The inventors found that the serum levels of TGF-beta, BMP9, angiopoietin-l, and angiopoietin-2 in wet AMD patients correlate with the responsiveness of the patients to the anti-VEGF therapy.

Accordingly, in an aspect the disclosure provides a method of predicting the responsiveness of a wet AMD patient to anti-VEGF therapy comprising

(1) determining the level of at least one marker protein selected from the group consisting of TGF-beta, BMP9, angiopoietin-1, and angiopoietin-2 in a blood, plasma or serum sample obtained from the patient, and
(2) predicting the responsiveness of the patient to the anti-VEGF therapy with reference to the level determined in step (1).

In an aspect, the disclosure provides a diagnostic agent for predicting the responsiveness of a wet AMD patient to anti-VEGF therapy comprising at least one antibody selected from the group consisting of anti-TGF-beta antibody, anti-BMP9 antibody, anti-angiopoietin-1 antibody, and anti-angiopoietin-2 antibody.

The disclosure enables predicting the responsiveness of a wet AMD patient to anti-VEGF therapy. The prediction may be useful for determining whether the patient should be treated with an anti-VEGF agent. When the patient has been treated with an anti-VEGF agent, the prediction may be useful for determining whether the treatment should be continued.

### BRIEF DESCRIPTION OF DRAWINGS

FIG. 1 shows the serum TGF-beta levels of the patients in control group, naive group, well response group, and refractory group (** p<0.01).

FIG. 2 shows the serum BMP9 levels of the patients in control group, naive group, well response group, and refractory group (** p<0.01).

FIG. 3 shows the serum angiopoietin-1 levels of the patients in control group, naive group, well response group, and refractory group (** p<0.05).

FIG. 4 shows the serum angiopoietin-2 levels of the patients in control group, naive group, well response group, and refractory group (** p<0.05).

### DETAILED DESCRIPTION

Unless otherwise defined, the terms used herein are read as generally understood by a skilled person in the technical fields such as organic chemistry, medicine, pharmacology, molecular biology, and microbiology. Several terms used herein are defined as described below. The definitions herein take precedence over the general understanding.

When a numerical value is accompanied with the term “about”, the value is intended to represent any value within the range of ±10% of that value. A numerical range covers all values from the lower limit to the upper limit and includes the values of the both limits. When a numerical range is accompanied with the term “about”, the both limits are read as accompanied with the term. For example, “about 20 to 30” is read as “20 t 10% to 30 i 10%.”

The term “anti-VEGF agent” as used herein means an agent that inhibits the function of a VEGF, including, e.g., VEGF inhibitors, VEGF receptor inhibitors, and nucleic acids that inhibit expression of a VEGF. Examples of anti-VEGF agents include ranibizumab, aflibercept, bevacizumab, and pegaptanib.

The term “the responsiveness of a wet AMD patient to anti-VEGF therapy” as used herein means the degree of the remission of the wet AMD patient caused by the anti-VEGF therapy. The degree of the remission may be determined by evaluating hemorrhage or leakage of blood components from CNV, the resulting accumulation of subretinal or intraretinal fluid (retinal edema or detachment of retinal pigment epithelium), or a symptom of wet AMD such as metamorphopsia, reduced visual acuity, central scotoma, defective color vision, or visual field defect. In an embodiment, the responsiveness is determined by evaluating the subretinal or intraretinal fluid or the reduced visual acuity.

The term “a wet AMD patient is responsive to the anti-VEGF therapy” as used herein means that the patient is remitted from wet AMD by the anti-VEGF therapy. For example, the patient is remitted within 12 months, preferably 6 months, more preferably 3 months from the start of the anti-VEGF therapy.

The term “remit” or “remission” as used herein means the cause of wet AMD is partially or completely reduced or removed, the progression of wet AMD is delayed or stopped, and/or a symptom of wet AMD is reduced, alleviated, ameliorated, or removed.

The term “patient” as used herein means an animal suffering from wet AMD. The animal may be a human or non-human animal. Examples of non-human animals include mice, rats, guinea pigs, rabbits, dogs, cats, monkeys, pigs, cattle, and horses, preferably mice, rats, guinea pigs, rabbits, dogs, and monkeys. In an embodiment, the patient is a human.

The term “marker protein” as used herein means a protein that is detected in a blood, plasma, or serum sample obtained from a wet AMD patient and is capable of indicating the responsiveness of the patient to the anti-VEGF therapy according to its level in the sample. The marker protein is at least one selected from TGF-beta, BMP9, angiopoietin-1, and angiopoietin-2 in the disclosure.

TGF-beta is a cytokine which suppresses angiogenesis (Int J Cancer. 2004; 108(2):251-7). The representative amino acid sequence of human TGF-beta (SEQ ID NO: 1) is deposited in GenBank (accession number P01137). BMP9 is a cytokine which promotes angiogenesis (PLoS One. 2012; 7(1):e30075.; J Cell Sci. 2010; 123(Pt 10):1684-92). The representative amino acid sequence of human BMP9 (SEQ ID NO: 2) is deposited in GenBank (accession number Q9UK05). Antagonism between TGF-beta and BMP9 is known.

Angiopoietin-1 is a growth factor which suppresses angiogenesis (Invest Ophthalmol Vis Sci. 2014; 55(4):2191-9). The representative amino acid sequence of human angiopoietin-1 (SEQ ID NO: 3) is deposited in GenBank (accession number Q15389). Angiopoietin-2 is a growth factor which promotes angiogenesis (J Clin Invest. 2012; 122(6):1991-2005). The representative amino acid sequence of human angiopoietin-2 (SEQ ID NO: 4) is deposited in GenBank (accession number 015123). Antagonism between angiopoietin-1 and angiopoietin-2 is known.

The four marker proteins may include deletion, substitution, or addition at one or several amino acids in their original sequences, for example, the amino acid sequences of SEQ ID NOs: 1 to 4, as long as their functions are maintained. The term “several amino acids” means preferably 2 to 7, more preferably 2 to 5, most preferably 2 to 3 amino acids. The amino acid substitution is preferably conservative substitution between similar amino acids.

The four marker proteins may comprise amino acid sequences having at least about 60%, preferably at least about 70%, more preferably at least about 80%, still more preferably at least about 90%, especially preferably at least about 95%, and most preferably about 97%, about 98%, or about 99% sequence identity with their original sequences, for example, the amino acid sequences of SEQ ID NOs: 1 to 4, as long as their functions are maintained. The sequence identity may be calculated with a program such as BLAST, for example, using BLAST with the parameters set to default values.

A blood, plasma or serum sample obtained from a wet AMD patient is used in the disclosed method. The blood sample may be obtained in a conventional manner, for example from a vein or artery of the patient. The plasma or serum sample may be prepared from the blood sample by any suitable methods known to those skilled in the art. The methods are not limited and may include any clinically acceptable steps, such as addition of an anticoagulant and centrifugation. The blood, plasma, or serum sample may be stored at a low temperature, for example in a freezer, during or after the preparation before the use. The blood, plasma, or serum sample may be diluted before the use as needed.

The levels of each marker protein may be determined with an immunoassay using an antibody capable of specifically binding to the marker protein. Examples of immunoassays include flow cytometry, radioimmunoassay (RIA), enzyme-linked immunosorbent assay (ELISA), western blotting, and immunohistological staining.

The term “antibody” as used herein means an affinity ligand based on an immunoglobulin scaffold. The antibody may be a monoclonal or polyclonal antibody of any origins. The antibody may be, e.g., a mouse, rat, rabbit, goat, or human antibody, or a chimeric antibody comprising sequences from different species, such as a partially humanized antibody, e.g., a partially humanized mouse antibody. The antibody may be produced by a conventional method in which a marker protein or its partial peptide having antigenicity is used as an immunogen. For example, a polyclonal antibody may be produced by immunizing an animal with an antigen. A monoclonal antibody may be produced by hybridoma technology. A commercially available antibody also may be used.

The antibody may be a fragment or derivative thereof that is capable of selectively interacting with the marker protein. Examples of antibody fragments or derivatives include Fab fragments, consisting of the first constant domain of the heavy chain (CH1), the constant domain of the light chain (CL), the variable domain of the heavy chain (VH) and the variable domain of the light chain (VL) of an intact immunoglobulin protein; Fv fragments, consisting of the two variable antibody domains VH and VL; single chain Fv fragments (scFv), consisting of the two VH and VL domains linked together by a flexible peptide linker; camelid heavy-chain dimers and single variable domains, and single domain scaffolds like e.g., the New Antigen Receptor (NAR) from the nurse shark and minibodies based on a variable heavy domain.

The antibody may be labeled. Those skilled in the art may select a suitable label. Examples of labels include fluorescent dyes or metals (e.g., fluorescein, rhodamine, phycoerythrin, fluorescamine), chromophoric dyes (e.g., rhodopsin), chemiluminescent compounds (e.g., luminal, imidazole), bioluminescent proteins (e.g., luciferin, luciferase), haptens (e.g., biotin), enzymes (e.g., peroxidase, alkaline phosphatase, beta-lactamase), radioisotopes (e.g., ³H, ¹⁴C, ³²P, ³⁵S, ¹²⁵I), and particles (e.g., metal particles such as gold), and fluorescent semiconductor nanocrystals (quantum dots). The different types of labels may be conjugated to the antibody using various chemistries known to those skilled in the art, e.g., amine reaction or thiol reaction. Other reactive groups, e.g., aldehydes, carboxylic acids and glutamine may also be used. Alternatively, the antibody may have no label and a labeled secondary antibody that binds to the primary antibody may be employed.

The antibody may be attached to a suitable support to provide an antibody array. Any support may be used irrespective of its shape or material, as long as it allows the attachment of the antibody. Examples of supports include a membrane, e.g. nylon membrane, beads, a glass support, a plastic support, or a metal support.

Once a level of a marker protein is determined, the responsiveness of the wet AMD patient to the anti-VEGF therapy is predicted with reference to the level. For example, the determined level is compared to a level of the marker protein in a blood, plasma, or serum sample from a wet AMD patient who is responsive to the therapy (responder) and/or a level of the marker protein in a blood, plasma, or serum sample from a wet AMD patient who is not responsive to the therapy (non-responder). Alternatively, the determined level may be compared to a predetermined standard level. The standard level may be an average level of the marker protein in blood, plasma, or serum samples from the responders and/or an average level of the marker protein in blood, plasma, or serum samples from the non-responders. Alternatively, the responsiveness may be predicted by comparing the determined level to a distribution of levels of the marker protein in blood, plasma, or serum samples from the responders and/or a distribution of levels of the marker protein in blood, plasma, or serum samples from the non-responders. The determined level is preferably compared on the basis of statistical significance.

The experiments described below proved that the blood levels of TGF-beta and angiopoietin-1 are higher in the responders than in the non-responders. This positive correlation enables predicting the responsiveness of a wet AMD patient to the anti-VEGF therapy. For example, when the level of at least one marker protein selected from the group consisting of TGF-beta and angiopoietin-1 in a wet AMD patient is evaluated to be relatively high, it may be predicted that the patient is more likely to be responsive to the anti-VEGF therapy.

The experiments described below proved that the blood levels of BMP9 and angiopoietin-2 are lower in the responders than in the non-responders. This negative correlation enables predicting the responsiveness of a wet AMD patient to the anti-VEGF therapy. For example, when the level of at least one marker protein selected from the group consisting of BMP9 and angiopoietin-2 in a wet AMD patient is evaluated to be relatively low, it may be predicted that the patient is more likely to be responsive to the anti-VEGF therapy.

Furthermore, the responsiveness of a wet AMD patient to the anti-VEGF therapy may be predicted more accurately with reference to the levels of two, three, or four of the marker proteins. In an embodiment, when the level of TGF-beta is evaluated to be relatively high and the level of BMP9 is evaluated to be relatively low, when the level of TGF-beta is evaluated to be relatively high and the level of angiopoietin-2 is evaluated to be relatively low, when the level of angiopoietin-1 is evaluated to be relatively high and the level of angiopoietin-2 is evaluated to be relatively low, or when the level of angiopoietin-1 is evaluated to be relatively high and the level of BMP9 is evaluated to be relatively low, it may be predicted that the patient is more likely to be responsive to the anti-VEGF therapy. In another embodiment, when the levels of both TGF-beta and angiopoietin-1 are evaluated to be relatively high, or when the levels of both BMP9 and angiopoietin-2 are evaluated to be relatively low, it may be predicted that the patient is more likely to be responsive to the anti-VEGF therapy. In another embodiment, when the levels of both TGF-beta and angiopoietin-1 are evaluated to be relatively high and the level of BMP9 is evaluated to be relatively low, when the levels of both TGF-beta and angiopoietin-1 are evaluated to be relatively high and the level of angiopoietin-2 is evaluated to be relatively low, when the level of TGF-beta is evaluated to be relatively high and the levels of both BMP9 and angiopoietin-2 are evaluated to be relatively low, or when the level of angiopoietin-1 is evaluated to be relatively high and the levels of both BMP9 and angiopoietin-2 are evaluated to be relatively low, it may be predicted that the patient is more likely to be responsive to the anti-VEGF therapy. In another embodiment, when the levels of both TGF-beta and angiopoietin-1 are evaluated to be relatively high and the levels of both BMP9 and angiopoietin-2 are evaluated to be relatively low, it may be predicted that the patient is more likely to be responsive to the anti-VEGF therapy.

Alternatively, the determined level may be compared to a predetermined cutoff value for the blood, serum or plasma level of the marker protein. For example, when the level of at least one marker protein selected from the group consisting of TGF-beta and angiopoietin-1 is not lower than a cutoff value, it may be predicted that the patient is more likely to be responsive to the anti-VEGF therapy. For example, when the level of at least one marker protein selected from the group consisting of BMP9 and angiopoietin-2 is not higher than a cutoff value, it may be predicted that the patient is more likely to be responsive to the anti-VEGF therapy.

The term “cutoff value”, also called as “threshold value”, means a value that serves as a reference to predict the responsiveness of a patient to the therapy. Preferably, the cutoff value enables high diagnostic sensitivity and high diagnostic specificity. The term sensitivity means true positive rate and the term specificity means true negative rate. For example, the cutoff value may be the blood, serum, or plasma level of a marker protein that is found in the responders at high positive rate and in the non-responders at high negative rate. Plural cutoff values may be given for one marker protein.

A cutoff value may be determined by any known statistic methods. Typically, a cutoff value may be determined by statistically processing the levels of a marker protein in blood, serum, or plasma samples obtained from the responders and the levels of the marker protein in blood, serum, or plasma samples obtained from the non-responders. A cutoff value may be determined by using software for statistical analysis, e.g., SAS Ver. 9.4.

For example, a cutoff value may be determined by receiver operating characteristic analysis (ROC analysis), which is conventionally used for evaluating effectiveness of diagnosing methods. The ROC analysis includes creating an ROC curve by plotting the sensitivity against the false positive fraction (FPF or false positive rate: 1—specificity) at various cutoff settings. When a diagnosing method is not effective at all, the ROC curve is a straight line connecting the lower left corner and the upper right corner. As the effectiveness gets higher, the ROC curve becomes round and gets closer to the upper left corner. When the effectiveness is 100%, the ROC curve passes through the left side and the upper side. In other words, when a diagnosing method enables both of the high sensitivity and high specificity, the ROC curve with an independent variable is close to the upper left corner. Accordingly, one way to determine a suitable cutoff value for a given diagnostic method is to create an ROC curve and identify a cutoff value that provides the point closest to the upper left corner on the ROC curve. An alternative way is creating an ROC curve and assuming an straight line whose area under the curve (AUC) is 0.05, and identifying a cutoff value that provides the point farthest from the straight line on the ROC curve. In other words, the latter way is calculating the values of (sensitivity+specificity−1) at various cutoff settings and identifying the cutoff value at which the Youden Index, the highest value of (.sensitivity+specificity−1), is given.

Typically, a cutoff value may be determined by obtaining blood, plasma or serum samples from the responders and the non-responders, determining the levels of each marker protein in the samples, calculating the values of the diagnostic sensitivity and specificity within the range of the determined levels, creating an ROC curve by using commercially available software for statistical analysis, and identifying the level at which the both values of the diagnostic sensitivity and specificity are as close to 100% as possible. Alternatively, a cutoff value may be based on diagnostic efficiency. The diagnostic efficiency means the proportion of the correctly diagnosed cases, including the cases in which the responders are correctly diagnosed as responders and the cases in which the non-responders are correctly diagnosed as non-responders, in all the diagnosed cases. The values of the diagnostic efficiency are calculated within the range of the determined levels of the marker protein and the cutoff value that provides the highest diagnostic efficiency may be identified.

When plural marker proteins are used, one cutoff value that allows to totally evaluate the levels of the plural marker proteins may be employed. For example, such cutoff value may be determined by conducting statistical analysis, e.g., logistic regression analysis, for plural marker proteins.

Once a cutoff value is identified, it may be optionally varied. In other words, a cutoff value may be freely set, depending on conditions, such as the combination of marker proteins, the method for determining and evaluating the level of marker proteins, the clinical purpose, and the desired sensitivity and specificity.

In an aspect, the disclosure provides a diagnostic agent for predicting the responsiveness of a wet AMD patient to anti-VEGF therapy comprising at least one antibody selected from the group consisting of anti-TGF-beta antibody, anti-BMP9 antibody, anti-angiopoietin-1 antibody, and anti-angiopoietin-2 antibody. The diagnostic agent is used for the method described above.

The antibody may be attached to a suitable support to provide an antibody array. Any supports usually used in the field may be used, including a membrane, e.g. nylon membrane, beads, a glass support, a plastic support, or a metal support.

Each component of the diagnostic agent may be provided separately or in a mixture with other component, as a solution in water or a suitable buffer such as phosphate buffered saline (PBS), or as a lyophilized product, in a suitable container.

The diagnostic agent may be provided in a diagnostic kit that further comprises a component or reagent necessary for determining the level of a marker protein. For example, the kit may comprise a labelled second antibody, a chromogenic substrate, a blocking solution, a washing buffer, an ELISA plate, or a blotting membrane.

In an aspect, the disclosure provides a method of predicting the responsiveness of a wet AMD patient to anti-VEGF therapy comprising

(1) obtaining a blood, plasma or serum sample from the patient,
(2) determining the level of at least one marker protein selected from the group consisting of TGF-beta, BMP9, angiopoietin-1, and angiopoietin-2 in the sample, and
(3) predicting the responsiveness of the patient to the anti-VEGF therapy with reference to the level determined in step (1).

In an aspect, the disclosure provides at least one antibody selected from the group consisting of anti-TGF-beta antibody, anti-BMP9 antibody, anti-angiopoietin-1 antibody, and anti-angiopoietin-2 antibody for use in predicting the responsiveness of a wet AMD patient to anti-VEGF therapy.

In an aspect, the disclosure provides use of at least one antibody selected from the group consisting of anti-TGF-beta antibody, anti-BMP9 antibody, anti-angiopoietin-1 antibody, and anti-angiopoietin-2 antibody for manufacturing a diagnostic agent for predicting the responsiveness of a wet AMD patient to anti-VEGF therapy.

In an aspect, the disclosure provides a method of treating wet AMD comprising administering an effective amount of an anti-VEGF agent to a wet AMD patient who has been predicted to be responsive to the anti-VEGF therapy by the diagnostic method of the disclosure. The term “treat” or “treatment” as used herein means a cause of wet AMD is reduced or removed, the progression of wet AMD is delayed or stopped, and/or a symptom of wet AMD is reduced, alleviated, ameliorated, or removed in a wet AMD patient. The effective amount of the anti-VEGF agent is not limited as long as it is sufficient for producing the desired medical effect. For example, from 0.0001 to 20 mg/eye is preferred, from 0.001 to 10 mg/eye is more preferred, from 0.01 to 5 mg/eye is still more preferred, and from 0.1 to 2.5 mg/eye is especially preferred.

In an aspect, the disclosure provides a method of treating wet AMD comprising

(1) obtaining a blood, plasma or serum sample from the patient,
(2) determining the level of at least one marker protein selected from the group consisting of TGF-beta, BMP9, angiopoietin-1, and angiopoietin-2 in the sample, and
(3) predicting the responsiveness of the patient to the anti-VEGF therapy with reference to the level determined in step (2),
(4) administering an effective amount of an anti-VEGF agent to the patient when the patient is predicted to be responsive to the anti-VEGF therapy in step (3).

Examples of anti-VEGF agents include ranibizumab, aflibercept, bevacizumab, and pegaptanib. Suitable dosage and dosage regimen of anti-VEGF agents are well known to those skilled in the art. For example, an anti-VEGF agent may be administered in accordance with directions for administrating the agent, for example those given in a package insert.

In an aspect, the disclosure provides a therapeutic agent for treating wet AMD comprising an anti-VEGF agent which is administered to a wet AMD patient who has been predicted to be responsive to anti-VEGF therapy by the diagnostic method of the disclosure.

In an aspect, the disclosure provides use of an anti-VEGF agent for treating wet AMD in a wet AMD patient who has been predicted to be responsive to anti-VEGF therapy by the diagnostic method of the disclosure.

In an aspect, the disclosure provides use of an anti-VEGF agent for manufacturing a therapeutic agent for treating wet AMD in a wet AMD patient who has been predicted to be responsive to anti-VEGF therapy by the diagnostic method of the disclosure.

In an aspect, the disclosure provides an anti-VEGF agent for use in treating wet AMD in a wet AMD patient who has been predicted to be responsive to anti-VEGF therapy by the diagnostic method of the disclosure.

For example, the disclosure provides the following embodiments;

[1] A method of predicting the responsiveness of a wet AMD patient to anti-VEGF therapy, comprising
(1) determining the level of at least one marker protein selected from the group consisting of TGF-beta, BMP9, angiopoietin-1, and angiopoietin-2 in a blood, plasma or serum sample obtained from the patient, and
(2) predicting the responsiveness of the patient to the anti-VEGF therapy with reference to the level determined in step (1).
[2] The method according to item [1], wherein the level of at least one marker protein is determined with an antibody specific for the marker protein in step (1).
[3] The method according to item [1] or [2], wherein the responsiveness is predicted by comparing the determined level to a cutoff value predetermined for the marker protein in step (2).
[4] The method according to item [3], wherein the patient is predicted to be responsive to the anti-VEGF therapy when the determined level of TGF-beta is higher than a cutoff value for TGF-beta.
[5] The method according to item [3], wherein the patient is predicted to be responsive to the anti-VEGF therapy when the determined level of BMP9 is lower than a cutoff value for BMP9.
[6] The method according to item [3], wherein the patient is predicted to be responsive to the anti-VEGF therapy when the determined level of TGF-beta is higher than a cutoff value for TGF-beta and the determined level of BMP9 is lower than a cutoff value for BMP9.
[7] The method according to item [3], wherein the patient is predicted to be responsive to the anti-VEGF therapy when the determined level of angiopoietin-1 is higher than a cutoff value for angiopoietin-1.
[8] The method according to item (3), wherein the patient is predicted to be responsive to the anti-VEGF therapy when the determined level of angiopoietin-2 is lower than a cutoff value for angiopoietin-2.
[9] The method according to item [3], wherein the patient is predicted to be responsive to the anti-VEGF therapy when the determined level of angiopoietin-1 is higher than a cutoff value for angiopoietin-1 and the determined level of angiopoietin-2 is lower than a cutoff value for angiopoietin-2.
[10] The method according to any one of items [3] to [9], wherein the cutoff value is determined by statistically processing the levels of the marker protein in blood, serum, or plasma samples from the wet AMD patients who are responsive to the anti-VEGF therapy and the levels of the marker protein in blood, serum, or plasma samples from the wet AMD patients who are not responsive to the anti-VEGF therapy.
[11] The method according to any one of items [1] to [10], wherein the patient has not received anti-VEGF therapy for treating wet AND.
[12] The method according to any one of items [1] to [10], wherein the patient is receiving or has received anti-VEGF therapy for treating wet AMD.
[13] The method according to any one of items [1] to [12], comprising administering an effective amount of an anti-VEGF agent to a wet AMD patient who is predicted to be responsive to the anti-VEGF therapy by the method according to any one of items [1] to [12].
[14] A diagnostic agent for predicting the responsiveness of a wet AMD patient to anti-VEGF therapy, comprising at least one antibody selected from the group consisting of anti-TGF-beta antibody, anti-BMP9 antibody, anti-angiopoietin-1 antibody, and anti-angiopoietin-2 antibody.

The entire contents of the documents cited herein are incorporated herein by reference.

The following example does not restrict or limit the invention and is for illustrative purposes only. The embodiments described above are non-limiting and may be modified without deviating from the scope of the invention as defined by the appended claims.

### Example 1

The correlation between the serum levels of biomarker candidates and the pathology of wet AMD was investigated for the following patient groups.

Group A: control (n=10)

Group B: naive (n=13)

Group C: well response (n=20)

Group D: refractory (n=14)

The inclusion and exclusion criteria for each group are summarized in the tables below.

TABLE 1

| | | Group | | | |
|---|---|---|---|---|---|
| No. | Inclusion criteria | A | B | C | D |
| 1 | Aged 65 or over and under 85 | Yes | Yes | Yes | Yes |
| 2 | having diagnosed cataract and | Yes | No | No | No |
| | undergoing a cataract surgery in | | | | |
| | near future | | | | |
| 3 | having wet AMD in at least one | No | Yes | Yes | Yes |
| | eye | | | | |
| 4 | having started anti-VEGF therapy | No | No | Yes | No |
| | (ranibizumab or aflibercept) 12 | | | | |
| | months before at the latest, | | | | |
| | having no subretinal fluid and/or | | | | |
| | intraretinal fluid (including | | | | |
| | symptoms such as hemorrhage and | | | | |
| | edema) detected by examination | | | | |
| | such as OCT, and having better | | | | |
| | visual acuity than before the | | | | |
| | start of the therapy | | | | |
| 5 | having started anti-VEGF therapy | No | No | No | Yes |
| | (ranibizumab or aflibercept) 12 | | | | |
| | months before at the latest, | | | | |
| | having received an anti-VEGF | | | | |
| | agent sequentially in recent | | | | |
| | three months, having subretinal | | | | |
| | fluid and/or intraretinal fluid | | | | |
| | (including symptoms such as | | | | |
| | hemorrhage and edema) detected by | | | | |
| | examination such as OCT, and | | | | |
| | having no better visual acuity | | | | |
| | than before the start of the | | | | |
| | therapy | | | | |
| 6 | undergoing anti-VEGF therapy in | No | Yes | Yes | Yes |
| | near future | | | | |

TABLE 2

| | | Group | | | |
|---|---|---|---|---|---|
| No. | Exclusion criteria | A | B | C | D |
| 1 | having diagnosed wet AMD in at | Yes | No | No | No |
| | least one eye | | | | |
| 2 | haying CNV in at least one eye | Yes | Yes | Yes | Yes |
| | due to a cause other than wet AMD | | | | |
| | (e.g., myopic CNV or injury) | | | | |
| 3 | having macular abnormality in at | Yes | Yes | Yes | Yes |
| | least one eye due to a cause | | | | |
| | other than wet AMD | | | | |
| 4 | having diagnosed retinal | Yes | Yes | Yes | Yes |
| | angiomatous proliferation (RAP) | | | | |
| | in at least one eye | | | | |
| 5 | having received an anti-VEGF | Yes | Yes | No | No |
| | agent through any administration | | | | |
| | route | | | | |
| 6 | having received an anti-VEGF | No | No | Yes | Yes |
| | agent through an administration | | | | |
| | route other than intravitreal | | | | |
| | injection | | | | |
| 7 | having undergone vitreous surgery | Yes | Yes | Yes | Yes |
| | in the eye to be observed | | | | |
| 8 | having undergone photodynamic | Yes | Yes | Yes | No |
| | therapy (PDT) in the eye to be | | | | |
| | observed | | | | |
| 9 | having undergone photodynamic | No | No | No | Yes |
| | therapy (PDT) in recent two years | | | | |
| 10 | having a complicated active | Yes | Yes | Yes | Yes |
| | inflammatory disease, including | | | | |
| | ocular infection and ocular | | | | |
| | allergy, in the eye to be | | | | |
| | observed, including eyelid | | | | |
| 11 | having undergone surgery selected | Yes | Yes | Yes | Yes |
| | from the followings in recent 90 | | | | |
| | days | | | | |
| | intraocular surgery in the | | | | |
| | eye to be observed, excluding | | | | |
| | vitreous surgery and PDT | | | | |
| | any intraocular surgery in the | | | | |
| | eye not to be observed | | | | |
| | any surgery due to a systemic | | | | |
| | disease | | | | |
| 12 | suffering from a malignant tumor | Yes | Yes | Yes | Yes |
| | or having suffered from a | | | | |
| | malignant tumor in recent five | | | | |
| | years | | | | |
| 13 | having participated in other | Yes | Yes | Yes | Yes |
| | clinical trial or clinical study | | | | |
| | in recent 30 days | | | | |
| 14 | being decided to be inadequate to | Yes | Yes | Yes | Yes |
| | the study by the attending | | | | |
| | physician | | | | |

Protocol for Immune Assay

The candidate biomarkers were 16 proteins which were suggested to be involved in angiogenesis. Serum samples were prepared using each 10 ml of blood samples collected from the patients. The levels of the biomarker candidates in the serum samples were measured using Aushon multiplex immunoassay platform (Aushon BioSystems, Billerica, Mass.). Antibody arrays were prepared by spotting capture antibodies specific to the 16 biomarker candidates on array plates. The samples were incubated for 2 hours on the antibody arrays and the plates were washed four times. Mixtures of biotinylated detection antibodies were added to each well, then the plates were incubated for 30 to 90 min and washed four times. The plates were then incubated with streptavidin-horseradish peroxidase conjugates for 30 min. All the incubation steps were carried out at room temperature with shaking at 200 rpm. The plates were washed again and chemiluminescent substances were added. The images of the plates were immediately acquired using Aushon Cirascan CCD Imaging System and the data were analyzed using Aushon Cirasoft Software.

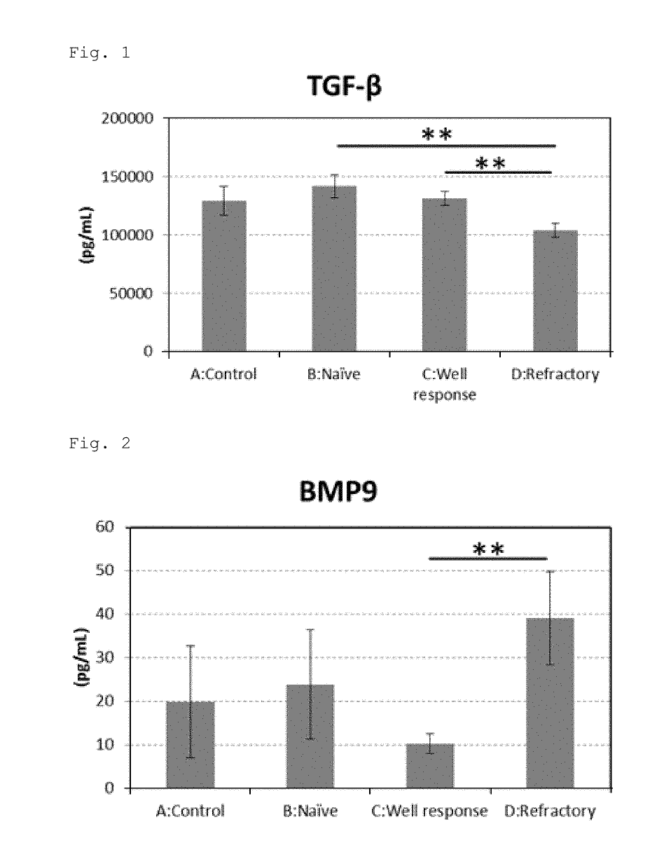

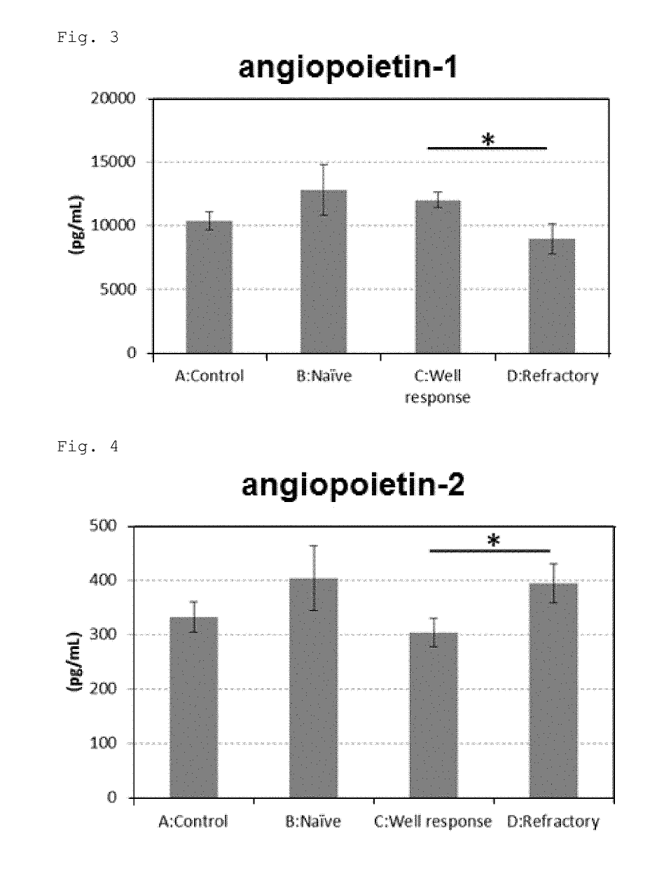

The results are shown in the following tables and FIGS. 1-4. The levels of TGF-beta, BMP9, angiopoietin-1, and angiopoietin-2 were significantly different between the well response group and the refractory group. The levels of TGF-beta and angiopoietin-1 were higher and the level of BMP9 and angiopoietin-2 were lower in the well response group.

TABLE 3

| | TGF-beta | | BMP9 | | angiopoietin-1 | | angiopoietin-2 | |
|---|---|---|---|---|---|---|---|---|
| Group | pg/ml | mean | pg/ml | mean | pg/ml | mean | pg/ml | mean |
| A: control | 95940.2 | 128926.8 | 4.5 | 19.8 | 7411.9 | 10380.1 | 207.0 | 331.9 |
| | 97144.4 | | 5.0 | | 8162.0 | | 238.8 | |
| | 104633.8 | | 5.3 | | 8464.3 | | 240.7 | |
| | 107036.9 | | 5.4 | | 8685.7 | | 269.0 | |
| | 116713.5 | | 5.5 | | 9462.5 | | 308.0 | |
| | 118855.1 | | 5.7 | | 11194.6 | | 385.8 | |
| | 122668.7 | | 8.6 | | 11506.3 | | 399.3 | |
| | 133271.9 | | 11.2 | | 12105.5 | | 420.6 | |
| | 171512.6 | | 11.4 | | 12163.3 | | 421.6 | |
| | 221510.8 | | 135.3 | | 14624.2 | | 428.0 | |

TABLE 4

| | TGF-beta | | BMP9 | | angiopoietin-1 | | angiopoietin-2 | |
|---|---|---|---|---|---|---|---|---|
| Group | pg/ml | mean | pg/ml | mean | pg/ml | mean | pg/ml | mean |
| B: naive | 104435.4 | 141272.0 | 4.7 | 23.8 | 5613.0 | 12782.4 | 208.6 | 403.5 |
| | 105639.8 | | 5.2 | | 6884.2 | | 223.8 | |
| | 111151.5 | | 5.7 | | 8359.0 | | 281.4 | |
| | 116483.9 | | 6.4 | | 9040.1 | | 290.8 | |
| | 127020.3 | | 7.1 | | 11218.7 | | 316.4 | |
| | 127804.1 | | 8.6 | | 11372.9 | | 332.1 | |
| | 128878.1 | | 9.8 | | 11479.4 | | 367.6 | |
| | 144808.0 | | 12.1 | | 12292.3 | | 367.7 | |
| | 147898.8 | | 14.2 | | 12586.6 | | 393.0 | |
| | 149281.0 | | 14.8 | | 12642.0 | | 423.5 | |
| | 155965.5 | | 15.8 | | 14438.2 | | 473.7 | |
| | 185276.0 | | 32.9 | | 15338.1 | | 511.4 | |
| | 231892.9 | | 171.9 | | 34906.5 | | 1056.0 | |

TABLE 5

| | TGF-beta | | BMP9 | | angiopoietin-1 | | angiopoietin-2 | |
|---|---|---|---|---|---|---|---|---|
| Group | pg/ml | mean | pg/ml | mean | pg/ml | mean | pg/ml | mean |
| C: well response | 84322.0 | 131263.7 | 4.3 | 10.2 | 7479.2 | 12003.5 | 111.8 | 303.6 |
| | 101438.6 | | 4.7 | | 8348.6 | | 147.5 | |
| | 107816.5 | | 5.3 | | 8943.6 | | 197.0 | |
| | 107855.2 | | 5.3 | | 9294.0 | | 201.6 | |
| | 110780.5 | | 5.9 | | 9320.1 | | 211.5 | |
| | 112161.4 | | 6.0 | | 10475.1 | | 233.0 | |
| | 114727.8 | | 6.1 | | 10696.3 | | 248.4 | |
| | 116311.0 | | 6.1 | | 10742.1 | | 255.0 | |
| | 118688.6 | | 6.2 | | 10789.9 | | 286.5 | |
| | 125840.4 | | 6.8 | | 10980.0 | | 291.7 | |
| | 132535.6 | | 6.9 | | 11883.5 | | 293.9 | |
| | 133213.1 | | 7.1 | | 12918.9 | | 307.9 | |
| | 138463.8 | | 7.2 | | 13293.2 | | 329.7 | |
| | 140793.9 | | 7.8 | | 13859.6 | | 332.1 | |
| | 142329.1 | | 8.0 | | 14005.5 | | 344.4 | |
| | 154820.2 | | 9.3 | | 14103.1 | | 372.1 | |
| | 155285.0 | | 9.4 | | 14333.7 | | 373.7 | |
| | 157602.9 | | 12.4 | | 15123.5 | | 401.0 | |
| | 170100.8 | | 35.2 | | 16487.9 | | 557.0 | |
| | 200188.3 | | 43.2 | | 16992.8 | | 576.3 | |

TABLE 6

| | TGF-beta | | BMP9 | | angiopoietin-1 | | angiopoietin-2 | |
|---|---|---|---|---|---|---|---|---|
| Group | pg/ml | mean | pg/ml | mean | pg/ml | mean | pg/ml | mean |
| D: refractory | 68648.4 | 103632.1 | 4.3 | 39.1 | 3226.1 | 8945.6 | 206.2 | 393.3 |
| | 74499.7 | | 6.3 | | 3728.2 | | 217.0 | |
| | 80128.6 | | 6.8 | | 5711.6 | | 220.8 | |
| | 84479.9 | | 9.0 | | 6790.6 | | 303.6 | |
| | 87914.6 | | 12.8 | | 7213.8 | | 313.6 | |
| | 97030.8 | | 13.4 | | 7464.5 | | 333.4 | |
| | 107789.4 | | 15.8 | | 7755.9 | | 403.8 | |
| | 108702.8 | | 27.8 | | 8476.2 | | 405.2 | |
| | 111708.6 | | 39.1 | | 8518.6 | | 435.3 | |
| | 116813.9 | | 44.0 | | 9254.7 | | 477.6 | |
| | 119080.9 | | 55.2 | | 9683.6 | | 502.2 | |
| | 126621.5 | | 76.3 | | 11634.5 | | 511.9 | |
| | 133592.8 | | 107.5 | | 17253.2 | | 537.5 | |
| | 135838.8 | | 128.6 | | 18327.2 | | 637.4 | |

Cut-off values were determined on the basis of the results of Groups C and D using SAS Ver. 9.4 software. Cut-off values were determined for each biomarker so that both of the diagnostic sensitivity and diagnostic specificity were as high as possible. More specifically, each cut-off value was determined so that the distance between the point where both of the sensitivity and specificity are 1 and the point given by the cut off value was the shortest, wherein the distance was calculated as “√((1-sensitivity)²+(1-specificity)²)”. Using the sample size above, the cut-off values for TGF-beta, BMP9, angiopoietin-1, and angiopoietin-2 were determined as 112161.4 pg/mL, 12.4 pg/mL, 9294.0 pg/mL, and 401.0 pg/mL, respectively.

The cut-off values gave the sensitivities and specificities shown in the table below. More reliable cut-off values could be found if a larger sample size is analyzed.

TABLE 7

| | sensitivity | specificity |
|---|---|---|
| TGF-beta | 75.0% | 64.3% |
| BMP9 | 90.0% | 71.4% |
| angiopoietin-1 | 85.0% | 71.4% |
| angiopoietin-2 | 90.0% | 57.1% |

Furthermore, the multivariate logistic regression analysis in which the four biomarkers are considered simultaneously gave the following model:

logit=−3.3987+(0.000063×TGF−beta level)−(0.0593×BMP9 level)+(0.000177×angiopoietin-1 level)−(0.0123×angiopoietin-2 level).

The value “logit” means log ((R)/(1−R)), wherein R is 1 for the well response group and 0 for the refractory group. When the cut-off value for the logit value was 0.86073, the distance between the point where both of the sensitivity and specificity are 1 and the point given by the cut off value was the shortest. When the cut-off value was used, the sensitivity and specificity were 85.0% and 92.9%, respectively. More reliable cut-off values could be found if a larger sample size is analyzed.

### INDUSTRIAL APPLICABILITY

The disclosure enables predicting the responsiveness of a wet AMD patient to anti-VEGF therapy. The prediction may be utilized for selecting therapeutic strategy suitable for the patient. For example, the anti-VEGF therapy may be applied or continued for the patients who are identified as being responsive to the therapy, whereas another therapy may be considered for the patients who are identified as being less responsive to the anti-VEGF therapy.

1. A method of predicting the responsiveness of a wet AMD patient to anti-VEGF therapy, comprising:
(1) determining the level of at least one marker protein selected from the group consisting of TGF-beta, BMP9, angiopoietin-1, and angiopoietin-2 in a blood, plasma or serum sample obtained from the patient, and
(2) predicting the responsiveness of the patient to the anti-VEGF therapy with reference to the level determined in step (1).

2. The method according to claim 1, wherein the responsiveness is predicted by comparing the determined level to a cutoff value predetermined for the marker protein in step (2).

3. The method according to claim 2, wherein the patient is predicted to be responsive to the anti-VEGF therapy when the determined level of TGF-beta is higher than a cutoff value for TGF-beta.

4. The method according to claim 2, wherein the patient is predicted to be responsive to the anti-VEGF therapy when the determined level of BMP9 is lower than a cutoff value for BMP9.

5. The method according to claim 2, wherein the patient is predicted to be responsive to the anti-VEGF therapy when the determined level of TGF-beta is higher than a cutoff value for TGF-beta and the determined level of BMP9 is lower than a cutoff value for BMP9.

6. The method according to claim 2, wherein the patient is predicted to be responsive to the anti-VEGF therapy when the determined level of angiopoietin-1 is higher than a cutoff value for angiopoietin-1.

7. The method according to claim 2, wherein the patient is predicted to be responsive to the anti-VEGF therapy when the determined level of angiopoietin-2 is lower than a cutoff value for angiopoietin-2.

8. The method according to claim 2, wherein the patient is predicted to be responsive to the anti-VEGF therapy when the determined level of angiopoietin-1 is higher than a cutoff value for angiopoietin-1 and the determined level of angiopoietin-2 is lower than a cutoff value for angiopoietin-2.

9. The method according to claim 1, wherein the patient has not received the anti-VEGF therapy for treating wet AMD.

10. The method according to claim 1, wherein the patient is receiving or has received anti-VEGF therapy for treating wet AMD.

11. A diagnostic agent for predicting the responsiveness of a wet AMD patient to anti-VEGF therapy, comprising at least one antibody selected from the group consisting of anti-TGF-beta antibody, anti-BMP9 antibody, anti-angiopoietin-1 antibody, and anti-angiopoietin-2 antibody.
